(19) United States
(12) Patent Application Publication
(10) Pub. No.: US 2014/0371814 A1
(43) Pub. Date: Dec. 18, 2014

(54) STIMULATION DEVICE AND METHOD OF USE THEREOF

(71) Applicant: DISRUPTIVE INNOVATIONS UNLIMITED, LLC, Atlanta, GA (US)

(72) Inventors: Paul SPIZZIRRI, Atlanta, GA (US); Chris Garvey, Roswell, GA (US)

(73) Assignee: DISRUPTIVE INNOVATIONS UNLIMITED, LLC, Atlanta, GA (US)

(21) Appl. No.: 14/305,823

(22) Filed: Jun. 16, 2014

Related U.S. Application Data
(60) Provisional application No. 61/835,356, filed on Jun. 14, 2013.

(51) Int. Cl. A61N 1/36; A61N 1/372; A61N 1/04

(52) U.S. Cl. CPC A61N 1/36014; A61N 1/0492; A61N 1/37264
USPC 607/48

(57) ABSTRACT

A stimulation device comprises a patch body having a battery and a processing unit. An adhering surface on an underside of the patch body is provided for attaching the patch body to a patient's skin. At least one electrode is disposed on or internal to the patch body. The processing unit is configured to communicate with a remote device and send electrical signals to the at least one electrode. The remote device may comprise an application to control the processing unit and a heart rate monitor may be in communication with the application. The application may automatically control the strength and timing of the electrical signals delivered to the at least one electrode based upon a monitored heart rate detected by the heart rate monitor.

### RELATED APPLICATION

The present disclosure claims priority to U.S. Provisional Patent Application 61/835,356, entitled, MUSCLE STIMULATION PATCH ELECTRODE AND WIRELESS CONTROL SYSTEM, filed Jun. 14, 2013, the full disclosure of which is incorporated herein by reference in its entirety.

### FIELD

The present disclosure relates generally to assemblies that comprise electrodes such as Electronic Muscle Stimulation (EMS) and/or Transcutaneous Electrical Nerve Stimulation (TENS) devices that are directed towards stimulating muscles and more particularly, systems that comprise such EMS/TENS devices that can be controlled wirelessly.

### BACKGROUND

Electrodes have been incorporated into EMS/TENS devices for some time so that they impart certain impulses to locations on a patient's skin. In turn, these various EMS/TENS devices can be configured and located on a patient for various applications. Such applications include muscle stimulation or strengthening, localized sensory stimulation, reduction of pain, and muscle memory training. Naturally, every unique application for EMS/TENS devices depends upon such factors as strength, number, and location of the EMS/TENS device(s) on a particular patient.

In turn, configuring a particular EMS/TENS device and/or locating the same on a particular patient requires a great deal of skill and patience from both the person locating and configuring as well as the patient having to remain still for relatively long periods of time. The EMS/TENS device may need to be manually adjusted by modifying the number or type of electrodes and all of the various settings related to how it stimulates the patient's skin. The mechanism that controls the EMS/TENS device may be directly connected through wires thereto so that mobility or quick-disconnection of the EMS/TENS device is impractical either from the control mechanism or from being installed on the patient herself. Therefore, there is a need to be able to easily and quickly locate, re-locate, configure and re-configure an EMS/TENS device(s) on a patient.

Moreover, in certain muscle stimulating applications, it has been difficult to predict how certain body parts or locations on a patient are affected by a given EMS/TENS configuration such as the patient's heart. For instance, the intensity, timing, or location of an EMS/TENS device(s) on a patient arranged to stimulate muscles may over-stimulate the patient so that the normal functioning of the patient's heart is unintentionally interrupted. To avoid potentially calamitous situation such as injury or even fatality, it is required to relocate, reconfigure, or even disengage the EMS/TENS device(s) from the patient. In current approaches, relocating, reconfiguring, or even disengaging EMS/TENS devices once installed on a patient is inefficient due to the time it consumes and the unnecessary risk it creates. Moreover, current approaches fail to predict or even measure the potentially calamitous situation once the EMS device is installed and in use.

Therefore, there is a need to provide a system with an EMS/TENS device that can be easily relocated, reconfigured so that intensity or timing of the EMS/TENS device can be adjusted, or even disengaged from a user without inflicting pain. There is also a need for a system with an EMS/TENS device that incorporates the capability to monitor a patient while the EMS/TENS device is being used to stimulate or otherwise affect a patient to avoid potentially harmful situations.

### SUMMARY

The following simplified summary is provided in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, a muscle stimulation device comprises a patch body and an adhesive surface on an underside of the patch body. The patch body comprises a battery and a processing unit. The adhesive or adhering surface attaches the patch body to a patient's skin. The adhesive or adhering surface may be any type of surface that attaches, adheres, or otherwise holds onto the skin of a patient. At least one electrode is disposed on or internal to the patch body to electrically stimulate the patient's skin, wherein the processing unit receives commands from a remote device independent from the muscle stimulation device, transmit information from to the remote device, and causes the at least one electrode to stimulate the patient's skin. There may be more than one electrode on the muscle stimulation device and in some embodiments, more than one remote device may be operatively coupled to the one or more muscle stimulation devices on the patient's skin.

The remote device may be a mobile device such as a smart phone, a table, a personal computer, a gaming console, or the like. The processing unit of the muscle stimulation device may comprise firmware so that the remote device can modify the firmware of the processing unit. This allows a user to modify settings associated with the firmware such as intensity of the electrodes, timing, patterns, duration, and overall resource management of the components controlled by the processing unit.

In some embodiments, an application is resident in the remote device for controlling the processing unit for the muscle stimulation device. The application may comprise signal authentication to prevent unauthorized control of the muscle stimulation device, for example, by an authorized third party. The application may control intensity and/or timing associated with the stimulation that is delivered by the at least one electrode to the patient. The application may be operatively coupled to a bio-feedback mechanism such as a heart rate monitor. The heart rate monitor may detect the heart rate of the patient, wherein the intensity and/or timing associated with the stimulation is adjusted or otherwise affected by the patient's detected heart rate.

A bio-feedback mechanism such as a heart rate monitor may be in communication with the application. In this embodiment, the application uses the monitored heart rate of the patient to automatically adjust and control the intensity and/or timing associated with the stimulation that is delivered by the at least one electrode to the patient's skin. The application may automatically control the intensity and/or timing based on the monitored heart rate relative to an associated predetermined threshold of the patient. The predetermined threshold may be.

The application may be configured to carry out the following: receive start and stop commands from the remote device; receive intensity control commands from the remote device; receive timing information from the remote device and the muscle stimulation device; receive status requests from the remote device; and respond to status requests with information received from the muscle stimulation device and the bio-feedback mechanism.

The battery of the muscle stimulation device may be lithium ion or made of a flexible polymer. A charging port on the patch body may be provided for charging the battery. The muscle stimulation device may further comprise an antenna to provide wireless communication between the processing unit of the muscle stimulation device and the remote device. An LED light may be provided on the patch body for indicating operational status to the user (e.g. a red light emitted from the LED light would indicate the muscle stimulation device is activated).

In other embodiments, a method of stimulating one or more muscles of a patient comprises the steps of: attaching muscle stimulation device on a predetermined location of a patient's skin; and wirelessly sending one or more commands to the processing unit of the muscle stimulation device from a remote device that is independent from the muscle stimulation device, wherein the one or more commands cause the at least one electrode to stimulate the skin.

The method may further comprise the step of adjusting a timing, an intensity, or a pattern of stimulation imparted by the at least one electrode to the skin through commands sent by the remote device. The method may comprise measuring a baseline heart rate of the patient with a heart rate monitor in communication with the remote device and then comparing the baseline heart rate with a present heart rate, wherein if a predetermined difference between the baseline and present heart rates is satisfied, then the timing, the intensity, or the pattern of stimulation imparted by the at least one electrode to the skin is further automatically adjusted. The method may comprise the step of wirelessly modifying firmware of the processing unit by communicating information from the remote device.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

FIG. 1 depicts one embodiment of a muscle stimulation device.

FIG. 2 illustrates a schematic overview of a system comprising the muscle stimulation device of FIG. 1 when positioned on the skin of a patient and in wireless communication with a remote device.

FIG. 3 illustrates a schematic overview of a system comprising the muscle stimulation device of FIG. 1 when positioned on the skin of a patient and directly connected to a remote device through a wire.

FIG. 4 depicts a flow diagram of programmable instructions comprised by an application utilized by a remote device of the system in FIG. 2.

FIG. 5 is a diagram that depicts certain conditions that must be met in an exemplary embodiment of the system of FIG. 2 or 3 to maintain the muscle stimulation device in a present state.

### DETAILED DESCRIPTION

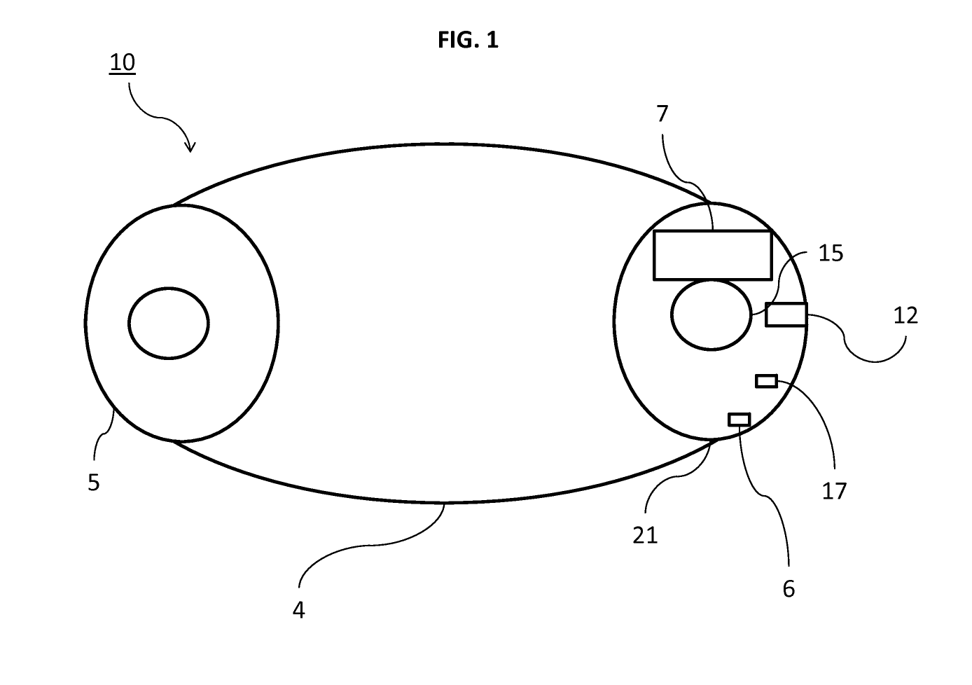

FIGS. 1-5 illustrate embodiments of the muscle stimulation device and systems incorporating the same so that a user or patient may wirelessly or directly control stimulation of predetermined location on a patient's skin such as muscles or nerves using one or more EMS devices and/or TENS devices in communication with one or more remote devices. This approach is particularly useful in any environment where a user, physician, trainer, patient or the like desires easy installation of an EMS/TENS device on a patient as well as easy adjustment of the device even after positioned and configured.

In some embodiments, there are two contemplated frequency groups: a first frequency group is a TENS frequency primarily directed at nerves rather than muscles. These frequencies are indicated for the relief of pain experienced through sensory neurons rather than for the toning, strengthening or conditioning of skeletal muscle tissue. By contrast, a second frequency group is directed towards electrical muscle stimulation through EMS devices (otherwise known as neuromuscular electrical stimulation). Optionally, it may be advantageous to alternate the first and the second frequency group during the same discrete treatment session. As a result, references herein to EMS devices and associated frequency, intensity, and other settings may more accurately be described as EMS/TENS rather than solely EMS. FIG. 1 depicts an EMS or TENS device 10 as described more particularly below. Device 10 may be a patch-like body 4 with one or more electrodes 15 positionable on a user's skin. Preferably, device 10 will fasten to the electrode using a snap-button attachment composed of metallic components that conduct electricity in order to transfer power from device 10 to animate and operate electrodes 15 to which device 10 is attached. Preferably, electrodes 15 are positioned on or near a muscle group or nerves to be exercised. In practice, the user 2 positions the patch body 4 and electrodes 15 directly to the skin at a predetermined location. Electrodes 15 may apply an automatically or manually timed stimulating pulse to the muscle group therebelow. This causes the muscle group to contract and intermittently relax, thereby causing the muscle to exercise.

Patch body 4 of device 10 may comprise at least one battery 5 designed to provide sufficient energy for other components comprised by device 10 such as processing unit 7, a charge port 12 configured to receive a charging wire and charge battery 5, and electrodes 15 that electrically stimulate muscle fiber in patient 2. Battery 5 may comprise at least one lithium ion button battery 5. An advantage of using a lithium ion battery 5 is that it allows for efficient energy storage as well as recharging capabilities in its current state. Optionally, a flexible lithium polymer battery may be used to reduce the number and size of rigid components of device 10. The one or more batteries 5 may be recharged by way of direct plug, removably attached or integrally formed flexible solar cells, or by inductive charging.

Processing unit 7 of device 10 may comprise low power processors with associated basic memory. The memory may comprise programmable instructions which in turn will control the different signal output states for device 10. Processing unit 7 preferably comprises a single on/off switch with an associated LED indicator light 17. Accordingly, when the device 10 is operating and therefore consuming resources from battery 5, the LED indicator light 17 will be turned on.

Processing unit 7 may be configured to receive input commands via the user from a predetermined digital transmission and communicate those commands to device 10 in order to electrically stimulate the patient. Accordingly, processing unit 7 may comprise a processor(s) and a unique control circuit configured to communicate and process instructions from remote device 3. Device 10 may further comprise an adhesive area 21 associated with each electrode 15, wherein the patient 2 or user may easily adhere the electrode 15 to a user-defined location so that the preferred muscle fiber is properly stimulated according to design needs and preferences.

The present device 10 may utilize approved EMS or TENS envelopes and power outputs to induce muscle contraction and release. Stimulation caused to a patient's skin by device 10 may be automatically or manually timed to provide exercise for a predetermined muscle or group of muscles and/or a predetermined stimulation regimen without conscious effort or instructions from a user beyond attaching the patch on the patient's 2 skin.

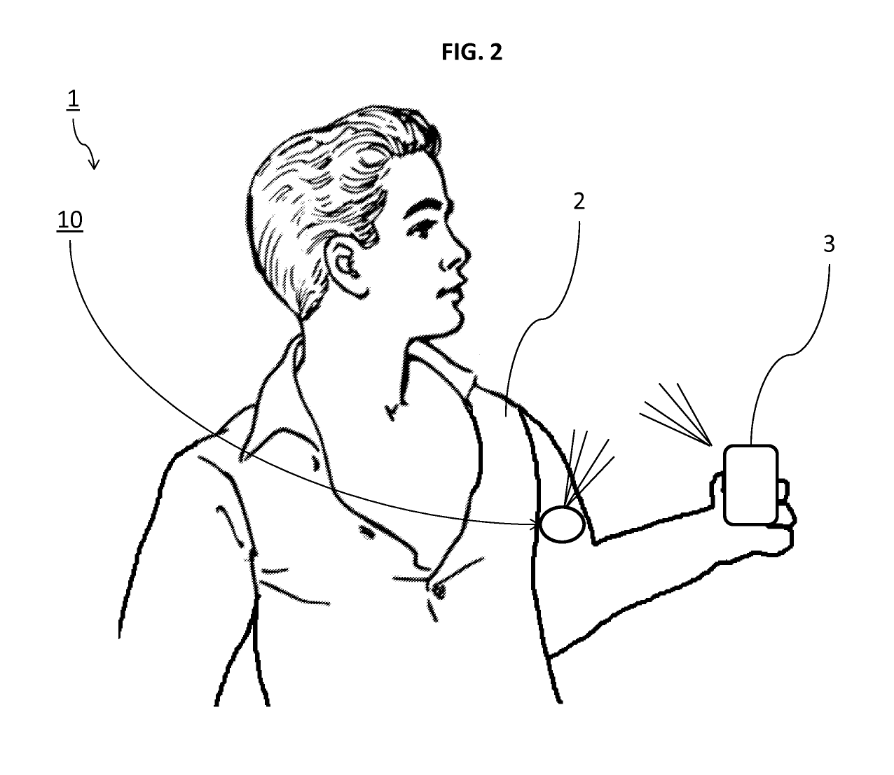

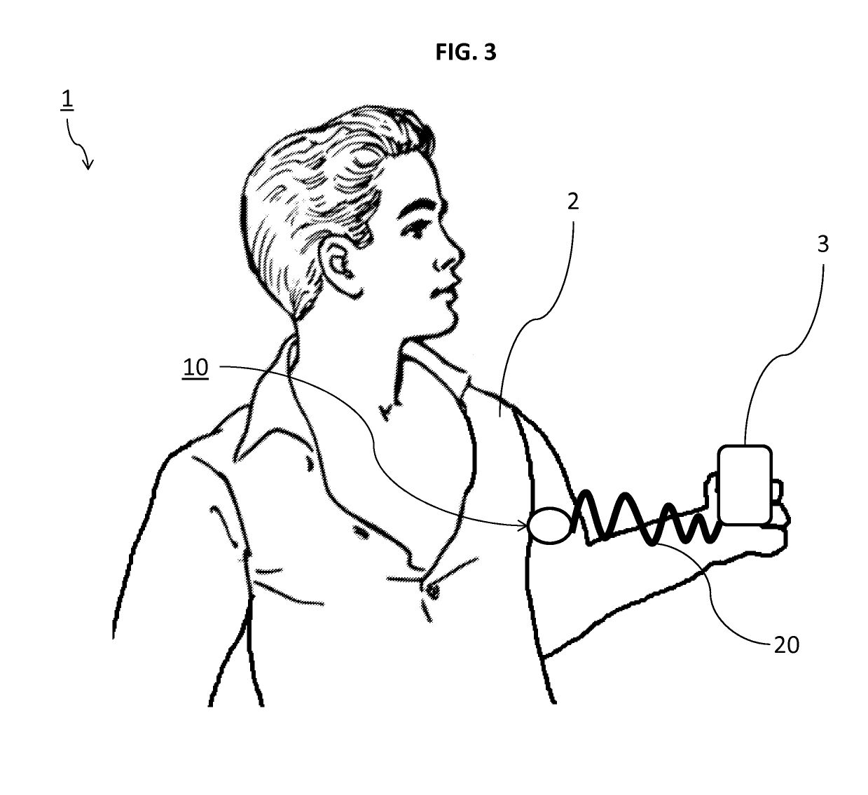

FIG. 2 depicts a system 1 where one or more devices 10 are installed and in use on a patient 2. When assembled, the present device 10 and/or system 1 may be both maximally flexible and minimally complicated. The patient 2 maintains control over the remote device 3 that is in operative wireless communication with device 10. It can be seen that remote device 3 is wirelessly coupled with device 10 so that device 10 is actuated and processing unit 7 can instruct electrodes 15 to electrically stimulate the pre-determined muscle group(s) of the patient 2. Alternatively, as seen in FIG. 3, remote device 3 may be configured to directly connect to device 10 through wires, bands, or the like 20. Wire 20 may be removably attachable to device 3 and/or device 10 depending on needs or preference. In FIG. 3, it can be seen that patient 2 has positioned device 10 on the upper bicep of his left arm. Patient 2 may position device 10 anywhere desirable and/or may additionally wirelessly couple and adhere any number of devices 10 elsewhere on the patient as needed or required. Accordingly, device 10 of FIG. 2 may be operable by Bluetooth, ANT+, or another digital standard. The digital standard selected for the digital control signal of device 10 may be producible and/or compatible with a standard consumer remote device 3 such as a smartphone, tablet, gaming console, personal computer or the like. In preferred embodiments, control signal transmission between devices 3 and 10 can be carried out by any suitable physical construction. In some embodiments, device 3 may comprise an application configured to control digital control signal transmission between devices 3 and 10. The application allows for digital inputs to the processing unit 7 from remote device 3. Control between the remote device 3 and the processing unit 7, wireless or otherwise, may be via Bluetooth, ANT+, RFID, or the like. Input from device 3 to processing unit 7 may be through voice commands from the user or patient. Voice commands may be processed through features integral to device 3 (e.g. Siri® on iPhone® or the like) or optionally through voice recognition features comprised by the application itself. Further, device 3 may be an iPad® or Android based tablet, wherein digital input between devices 3 and 10 may include various forms of telecommunication such as text messaging, email and voice calls.

Preferably, a control link between device 10 and device 3 will comprise signal authentication, to prevent unauthorized control of device 10. This functionality will preferably also allow for relatively low energy packeted information to be sent to device 10, in order to command processing unit 7 to start, stop, or switch predetermined EMS or TENS patterns, intensity, timing or the like.

The application may be resident in remote device 3 and may be designed to work with the control circuit of processing unit 7. This is particularly advantageous since it means that the application may be downloaded onto and stored in device 3 from any location in communication with, for example, the internet. This provides the added advantage of being able to re-configure firmware associated with various settings of processing unit 7 of device 10 such as, but not limited to, timing, intensity, duration, or resource management without having to disengage, remove, or otherwise manipulate device 10.

For example, an update to the firmware that more efficiently manages resources can lead to smaller batteries, increased number of uses per battery 5, or fewer required charges of battery 5 between uses. Revisions in the firmware of device 10 may further lead to increased accuracy of intensity or latency of a particular electrode 15 or its timing which in turn leads to increased safety and efficacy for a particular treatment regimen associated with device 10.

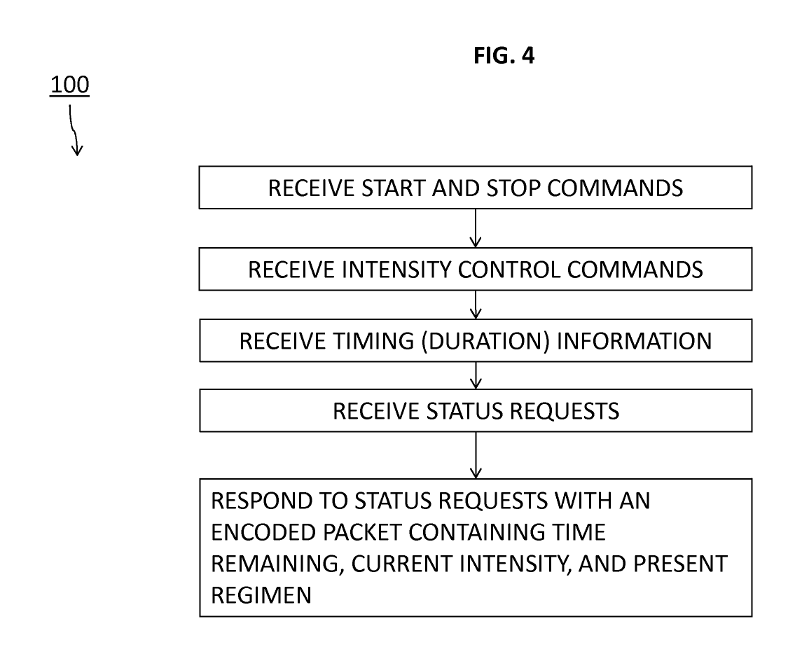

FIG. 4 depicts a flow diagram of programmable instructions 100 that may be permanently stored in nonvolatile memory or otherwise comprised by the application, wherein the processing unit 7 of device 10 is instructed to:
1. Receive Start and Stop commands;
2. Receive intensity control commands;
3. Receive timing (duration) information;
4. Receive status requests; and
5. Respond to status requests with an encoded packet containing time remaining, current intensity, and current regimen.

The application comprised by device 3 may preferably be able to utilize multiple security modes available from Bluetooth or other wireless transmission standards. Further, such capabilities may be integrally formed into the antenna in device 10 or may be removably attachable to the same. The application of device 3 may be able to control two or more devices 10 effectively and simultaneously, and may also be configured to process information from other third party devices.

In practice, once device 10 is powered on, light 17 may illuminate. From there, the user or patient 2 may activate the application from remote device 3. Once the application is activated, the application may automatically scan and locate device(s) 10. There may be one or more devices 10 positioned on a patient, depending on needs and preference. Once all devices 10 are located by application of device 3, the user or patient will be able to view the devices on a display medium in communication with the application. The user or patient can select or define the type of stimulation they will utilize at this step. In some embodiments, the application may comprise pre-defined regimens including parameters such as timing, intensity, pattern, or duration. Once a particular regimen is defined or selected for all positioned devices 10 and associated electrodes 15, the patient's 2 skin will begin to be stimulated.

To that end, patch body 4 of device 10 may further comprise antenna 6 for communication between devices 3 and 10. Antenna 6 may be etched onto or otherwise connected to a pliable plastic. This allows for antenna 6 to be sewed onto the patch body 4. Attaching antenna 6 to patch body 4 in this manner allows the patch body 4 to remain as flexible as possible, while still allowing for a relatively large antenna 6 area, should this prove necessary. In other embodiments, antenna 6 may be removably attachable by fastener, snap on, or hook and look fasteners so that antenna 6 can be modified or re-positioned as needed or desired for a particular design.

Optionally, a timing circuit may be included with processing unit 7. This timing circuit may use a simple hardwired clock with one or more transistors operating as switches operable by processor unit 7. The one or more transistors may control which Resistor/Capacitor (RC) timing is selected, and can ultimately control the nature of the signal being output by device 10 to the patient 2.

A clock signal of the timing circuit can optionally be fed into two different waveform generators. A first waveform generator may be an envelope generator. In this embodiment, the envelope generator provides the overall shape of the pulse train. A second waveform generator may be a square wave generator, which may output a relatively consistent square wave pattern. The first and/or second waveforms can be fed into a mixing circuit which can combine each waveform into the signal that causes device 10 to electrically stimulate the pre-determined location of the patient.

In other embodiments, the signal output of device 10 may optionally be fed through a power control unit, which may be directly controlled by processing unit 7. This allows for several pre specified power levels. For instance, a low power setting might be included in order for new users to acclimate themselves to a specific stimulation or pulse. The signal output of device 10 may then be sent to electrodes 15. Because electrodes 15 may be placed on the underside of an adhesive pad on the patch body 4, stimulations imparted by electrodes 15 to the patient 2 cause muscle contractions in the area directly below or adjacent to the patch 4.

In preferred embodiments, the present control system may provide a bio-feedback mechanism such as a heart rate monitor or the like. In practice, antenna 6 may be bi-directional. Bi-directional antenna 6 allows for feedback to device 3 by both visual references perceived by device 3 and associated features such as visual and our audio sensory mechanisms. Such feedback can include verbal feedback and other information sensed by device 3 such as feedback from a pedometer, heart rate monitors, a GPS device, a fitness recordation device, temperature gauges, or sweat gauges that sample electrolyte composition. Application of device 3 can be configured to analyze received feedback from any of the foregoing device in order to automatically adjust the operation of device 10. For example, because it is known that heart rate increases with certain sensations such as pain, the present control system can optionally sense if the stimulation is painful for a patient 2 as a result of a predetermined increase in heart rate. If the stimulation, for example, is detected to exceed the predetermined increase in heart rate such that it is deemed painful, the associated intensity of the EMS signal delivered by electrode(s) 15 to a patient 2 can be automatically reduced by the control system. Alternatively, the intensity may be manually adjustable.

In one exemplary method, the patient's heart rate is measured for a predetermined period of time such as 15 seconds in order to provide a baseline heart rate. The heart rate can then be monitored by the heart rate monitor and analyzed by comparing changes in present heart rate to the baseline heart rate established during the measurement of the predetermined period of time. For example, if the patient's heart rate increases after a second predetermined period of time such as 3 seconds, then the intensity of the pulse imparted by electrodes 15 of device 10 to the patient 2 could be adjusted or reduced as needed or desired. In some embodiments, should the heart rate being measured exceed the baseline heart rate measurement by a certain factor (e.g. 11% and adjustable by user), then device 3 may automatically issue a Stop command to device 10. A Stop command may cause the present routine to pause. Device 10 may stop issuing stimulation, but may also stop the clock on the routine, waiting for either a Start command or new routine command to begin again. Further, it is contemplated that the intensity can be adjusted or reduced by one unit of intensity measure periodically (e.g. every three seconds). These automatic adjustments or reductions can preferably be made subject to manual override.

If the user has never used the application before, the application will prompt the user to select or deselect heart rate monitoring. Should the user select heart rate monitoring, device 3 will inform the user to begin moving about during the routine. The heart rate monitor then monitors the patient for a predetermined period of time (e.g. 15 seconds) to establish the baseline heart rate measurement. This is particularly advantageous as instead of relying on a single average heart rate, system 1 may instead periodically measure heart rate intervals for every predetermined period of time (e.g. 15 seconds) and calculate the heart rate measurement using the maximum and minimum heart rate to compute a heart rate measurement considered the user's “baseline.” After establishing the heart rate baseline, the device 10 maybe enabled with the user defined or automatic routine settings, and the application will begin monitoring the heart rate of the user.

In accordance with the present disclosure, the control circuit of processing unit 7 may be designed such that relatively slight increases in heart rate do not automatically reduce or adjust the intensity of device 10 too quickly or result in a Stop command. The problem to avoid is a patient who quickly interrupts use of device 10, for example, by jumping up to answer a ringing doorbell, and immediately shutting down device 10. Quickly turning off device 10 can lead to injury, increased pain, or the like. To resolve this problem, the control circuit may be designed such that only relatively large changes in heart rate—preferably over relatively prolonged periods of time—may significantly vary the treatment regimen.

Remote device 3 may primarily control the device 10 but may also comprise additional features. The previously described application can wirelessly issue commands to the device 10 via wireless protocols such as Bluetooth or the like. These issued commands control parameters such as the time when the device 10 is active or the intensity of the stimulation ultimately imparted by the device 10 to the patient 2. Due to the fact that the application of device 3 may lose connectivity with the device 10 permanently or intermittently (e.g. battery in the device 3 runs out of energy), the device 10 can be configured to operate in a semi-autonomous mode. Upon start up, the device 10 may operate in a standby mode, meaning, the device 10 performs no regimen and paging for wireless protocols such as Bluetooth.

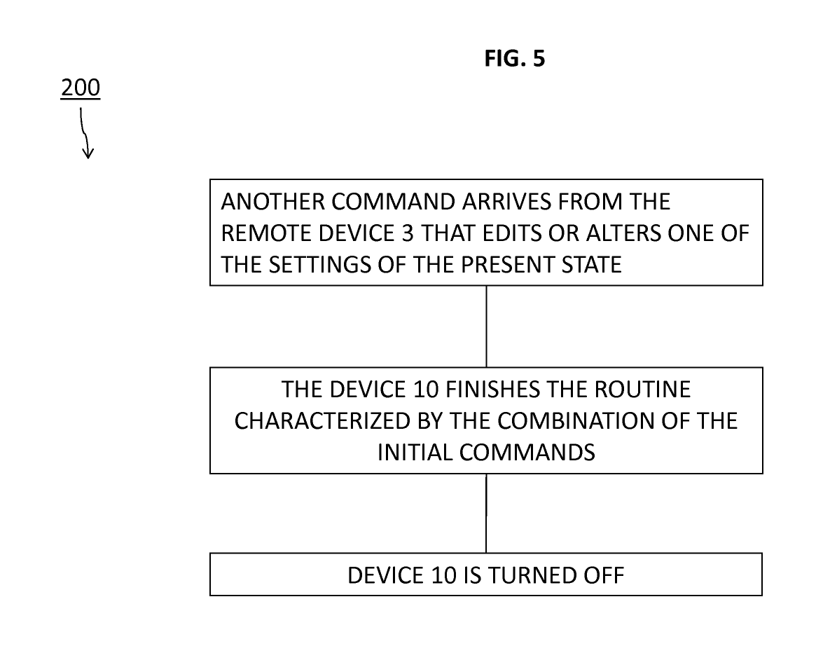

When the application of device 3 instructs to connect with the device 10, the device 10 may wait for a predetermined combination of instructions 100 (this combination of instructions 100 hereon referred to as a routine). The device 10 may then store each of these pieces of information as its present state. As depicted in FIG. 5, device 10 may maintain its present state unless one of three conditions is satisfied.
1. Another command arrives from the remote device 3 that edits or alters one of the settings of the present state;
2. The device 10 finishes the routine characterized by the combination of the initial commands; or
3. The device 10 is turned off.

Should another command arrive, the device 10 may begin operating on the new, instructed routine as quickly as possible. Should the routine finish, the device 10 could return to a semi-rest mode, performing no action but remaining connected wirelessly to the remote device 3.

In some embodiments, if the device 10 is turned off while it is still running a routine, the routine which is stored in nonvolatile or volatile memory will be lost. Upon turning back on device 10, device 10 will automatically enter the previously described semi-rest mode. Note that the wireless connection may have to be reestablished to the extent it is desired by the user or patient to activate device 10 from the semi-rest mode. In those embodiments where device 10 achieves wireless connectivity via Bluetooth, device 10 can be activated from a semi-rest mode by switching to third mode, authenticated, Bluetooth security and using a predetermined PIN which devices 3 and 10 and the associated application will agree upon when Bluetooth connection is established.

In some embodiments, should the device 10 finish its routine while in the third mode Bluetooth security, device 10 will revert to the semi-rest mode completely. The application comprised by device 3 may further comprise a graphical user interface (GUI) allowing the user to select the regimen and/or associated intensity, adjust the duration of each, and provide capability to automatically or manually start or stop the current routine. The GUI may also provide access to wireless transmission settings (e.g. Bluetooth settings). This allows a user to easily integrate third party devices such as additional mobile devices, tablets, gaming consoles, personal computers, or the like. Moreover, this allows a user to customize and more easily use specific user settings as well as provide access to a history of routines.

Upon issuance of a routine to device 10, the routine information and the PIN associated with application and device 10 can be time stamped and stored in non volatile memory of the processing unit 7 of device 10 or remote device 3. Should the remote device 3 that executes the application be turned off, then upon restarting of the application, the application may automatically check for a stored time stamped routine. If the application determines that a stored time stamped routine exists, the application will attempt to reconnect to device 10 using the stored PIN. This allows the application to securely reconnect with the correct device 10. The application may then poll for routine information, analyze and compare this polled information with the time stamped routine. Should the polled routine information and time stamped routing match in regimen and intensity, the application may adjust the remaining duration to the polled routine information.

The application may further comprise features based on the availability of information from third party devices as previously described. Should a third party full function fitness monitor such as Nike+, Fitbit Flex, or the like be available, the application may be configured to communicate with the third party fitness monitor as to completed routines to be included in their respective calculations for physical exertion or the like. The application may also request information from device 10 itself regarding physical motion and use this to adjust the heart rate threshold associated with the previously described baseline heart rate measurement. In some embodiments, this reduces the number of instances that false Stop commands are received by the application.

Many alterations and modifications may be made by those having ordinary skill in the art without departing from the spirit and scope of the embodiments disclosed and described herein. Therefore, it is understood that the illustrated and described embodiments have been set forth only for the purposes of examples and that they are not to be taken as limiting the embodiments as defined by the following claims. For example, notwithstanding the fact that the elements of a claim are set forth below in a certain combination, it must be expressly understood that the embodiments include other combinations of fewer, more or different elements, which are disclosed above even when not initially claimed in such combinations.

The definitions of the words or elements of the following claims are, therefore, defined in this specification to not only include the combination of elements which are literally set forth. It is also contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination(s).

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what incorporates the essential idea of the embodiments.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A muscle stimulation device, comprising:
a patch body comprising a battery and a processing unit;
an adhering surface on an underside of the patch body for attaching the patch body to a patient;
at least one electrode disposed on or internal to the patch body;
wherein the processing unit is configured to communicate with a remote device and cause the at least one electrode to electrically stimulate the patient.

2. The muscle stimulation device of claim 1, wherein the remote device is a mobile device.

3. The muscle stimulation device of claim 1, wherein the processing unit comprises firmware, and wherein the remote device is configured to modify the firmware of the processing unit.

4. The muscle stimulation device of claim 2, wherein an application resident in the mobile device controls the processing unit.

5. The muscle stimulation device of claim 4, wherein the application further comprises signal authentication to prevent unauthorized control of the muscle stimulation device.

6. The muscle stimulation device of claim 4, wherein the application controls an intensity or an interval associated with an electrical stimulation that is delivered by the at least one electrode to the patient's skin.

7. The muscle stimulation device of claim 4, further comprising:
a bio-feedback mechanism in communication with the application, wherein the application automatically controls an intensity or an interval associated with an electrical stimulation that is delivered by the at least one electrode to the patient based upon a heart rate detected by the bio-feedback mechanism.

8. The muscle stimulation device of claim 7, wherein the application automatically controls the intensity and the interval based on the monitored heart rate relative to an associated predetermined threshold of the patient.

9. The muscle stimulation device of claim 7, wherein the bio-feedback mechanism is a heart rate monitor resident in the remote device.

10. The muscle stimulation device of claim 8, wherein the application is configured to:
receive, start, and stop commands from the remote device;
receive an intensity control command from the remote device;
receive timing information from the remote device and the muscle stimulation device;
receive a status request from the remote device; and
respond to the status request with information received from the muscle stimulation device and the bio-feedback mechanism.

11. The muscle stimulation device of claim 1, wherein the battery is a lithium ion battery.

12. The muscle stimulation device of claim 11, wherein the lithium ion battery is made of a flexible polymer.

13. The muscle stimulation device of claim 1, wherein the processing unit comprises an antenna to provide wireless communication between the processing unit and the remote device.

14. The muscle stimulation device of claim 1, further comprising:
a light for indicating operational status.

15. The muscle stimulation device of claim 1, further comprising:
a charging port on the patch body for charging the battery.

16. A method of stimulating muscles of a patient, comprising:
attaching muscle stimulation device on a predetermined location of a patient's skin, the muscle stimulation device comprising:
a patch body comprising a battery and a processing unit;
an adhering surface on an underside of the patch body for attaching the patch body to the skin;
at least one electrode on or disposed in the patch body;
wirelessly sending one or more commands to the processing unit from a remote device that is independent from the muscle stimulation device, wherein the one or more commands cause the at least one electrode to stimulate the skin.

17. The method of claim 16, further comprising:
adjusting a timing, an intensity, or a pattern of stimulation imparted by the at least one electrode to the skin through commands sent by the remote device.

18. The method of claim 17, further comprising:
measuring a baseline heart rate of the patient with a heart rate monitor in communication with the remote device;
comparing the baseline heart rate with a present heart rate, wherein if a predetermined difference between the baseline and present heart rates is satisfied, then the timing, the intensity, or the pattern of stimulation imparted by the at least one electrode to the skin is further automatically adjusted.

19. The method of claim 16, further comprising:
wirelessly modifying firmware of the processing unit by communicating information from the remote device.
